(19) United States
(12) Patent Application Publication
(10) Pub. No.: US 2018/0117056 A1
(43) Pub. Date: May 3, 2018

(54) CONTROL OF PARASITIC INFECTION IN ANIMALS

(71) Applicant: CROPMARK SEEDS LIMITED, Christchurch (NZ)

(72) Inventors: Brian James PATCHETT, Christchurch (NZ); Nicholas Evan CAMERON, Selwyn (NZ)

(73) Assignee: CROPMARK SEEDS LIMITED, Christchurch (NZ)

(21) Appl. No.: 15/565,457

(22) PCT Filed: Apr. 8, 2016
(86) PCT No.: PCT/NZ2016/000001

§ 371 Date: Oct. 10, 2017

(30) Foreign Application Priority Data
Apr. 9, 2015 (NZ) 706869

(51) Int. Cl. A61K 31/5386; A61P 33/00

(52) U.S. Cl. CPC A61K 31/5386; A61P 33/00

(57) ABSTRACT

The invention relates to the use of alkaloid lolines to reduce parasitic infection in livestock and especially in ruminants, particularly sheep. The loline alkaloids are produced by and found in, grasses infected by endophytes. These grasses can be sown in areas where ruminants graze. Consumption of the grass supports the accumulation of loline alkaloids in the rumen of the animals and leads to a lower rate of infection by internal parasites and hence healthier livestock. The loline alkaloids may be produced by a number of endophytes present in a range of suitable grasses. The invention also provides a range of grasses which are suitable for the treatment of internal infection by parasites in livestock, especially ruminants and more especially sheep.

### FIELD OF THE INVENTION

The invention relates to the use of loline alkaloids for the treatment of parasites in animals and more particularly to the use of these compounds in the treatment of internal parasites in farm animals.

### BACKGROUND TO THE INVENTION

Intestinal parasites are a major cause of concern in livestock in New Zealand.

Nematode parasites were probably introduced with the first sheep imported into New Zealand and have been a major limiting factor to sheep production for more than 100 years. The dynamics of nematode infection are a consequence of complex inter-relationships of sheep, their husbandry and the prevailing climate. Traditional methods of control have been based on the use of anthelmintics but increasing incidence of resistance by parasites is challenging the ability of producers to maintain high levels of productivity. Parasite resistance to anthelmintics and the desire for residue free food products has resulted in searches for alternative means of parasite control. Alternative technologies include vaccines, immunomodulation, bio control agents such as nematophagous fungi and plants, and molecular techniques.

Nematophagous fungi have been investigated as a possible effective bio control of intestinal parasites. These fungi invade dung on pastures before growing, trapping and killing parasite larvae as they develop in the faeces. The species most closely , examined in New Zealand is Duddingtonia flagrans. Unfortunately, although the efficacy of the fungus was demonstrated in vitro, these results did not translate into reduced faecal egg counts (FECs) or improved animal performance in the field.

Pastures modify the microclimate that may directly affect larval development and survival, effect egg and larval predators and pathogens and alter the rate of faecal decomposition. There have been a number of investigations to determine the effect of grasses and herbs on faecal egg count, larval survival and worm burden. Differences between grasses on these parameters have been observed. Plant morphology can alter larval migration.

Plantain (Plantago lanceolata) contains phenolic glycosides and sainfoin (Onobrychis viciifolia) contains flavanol glycosides which have also been shown to have anti-parasitic and/or immunity enhancing properties. Many of these plants are, however, difficult to establish, are poorly adapted to regular grazing, and have not been widely adopted despite considerable promotion by industry and the scientific community.

Temperate grasses, in contrast, grow readily in the cool moist climates enjoyed over most of New Zealand. Since the discovery of the relationship between endophyte, fescue toxicosis and ryegrass staggers, endophyte containing grasses have been viewed as toxic to animals and have not been pursued as a possible source of anthelmintic agents.

Evidence for an effect of loline alkaloids on nematodes is limited to reports on plant nematodes. Endophyte alkaloids have generally been considered as deleterious to livestock.

The effect of loline alkaloids on intestinal nematodes remains unresolved. The presence of ergot alkaloids in experiments involving tall fescue has seriously compromised opportunities to understand the role of loline alkaloids. This is accentuated in animal experiments because of the extreme toxicity of ergot alkaloids to livestock.

### OBJECT OF THE INVENTION

It is therefore an object of the invention to provide a method of treatment of livestock by using loline alkaloids to treat infection of the intestinal tract by parasitic organisms, or to at least provide the public with a useful option.

### SUMMARY OF THE INVENTION

The invention provides the use of loline alkaloids in the control of internal parasitic infestation in livestock.

Preferably the livestock includes ruminants, especially goats, cattle and sheep. More preferably the livestock includes sheep.

Preferably the internal parasites controlled include nematodes and especially intestinal nematodes.

More preferably the parasites treated include: abomasum worms, duodenum worms, large intestine worms and small intestine worms.

More preferably the intestinal parasites include Teladorsagia circumsincta and Trichostrongylus colubriformis.

The lolines used include any compound with the following chemical structure:

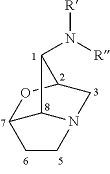

with R′ and R″ denoting variable substituents that can include methyl, formyl and acetyl groups giving rise to different forms of loline including N-formyl loline (NFL); N-acetyl loline (NAL); N-methyl loline (NML); and N-acetyl norloline (NANL).

In particular, the lolines included within the scope of this specification include NFL (N-formyl loline), NAL (N-acetyl loline), NANL (N-acetyl norloline) and NML (N-methyl loline)

The lolines can exist in any grass to limit the population and provide control of intestinal parasites in ruminants.

The invention provides the use of a plant species containing loline alkaloid for the treatment and/or control of internal parasitic infestation in livestock, particularly in sheep.

The plant species could include any selected from the group comprising: Lolium perenne, Lolium multiflorum, Lolium temulentum, Festuca orundinacea, Festuca pratensis, and any hybrid combinations of these species, Bromus auleticus, Adenocarpus species, Argyreia mollis.

Also included is any grass, cereal or forage containing loline alkaloids, or any endophyte x grass combination including the endophytes Epichloe coenophialum (=Neotyphodium coenophialum), E. occultans, E siegelli, E. pampeanum, E. uncinatum (new naming Epichloe=Neotyphodium) producing lolines in forage roots and seed, which are useful in the treatment of intestinal parasites in ruminants.

In particular, the invention provides the use of a plant extract containing loline alkaloids and the use of plants containing loline alkaloids, in the treatment of livestock for internal parasitic infestation, in livestock.

The invention also provides the use of a composition containing loline alkaloids in the treatment and/or control of internal parasitic infestation in livestock, particularly sheep.

In particular, the invention provides the use of an endophyte and in particular the endophyte Epichloe uncinata (syn. Neotyphodium uncinatum) to control intestinal parasites in livestock. However, the invention also provides the use of other endophytes including but not limited to Epichloe spp. (syn. Neotyphodium species), E. seigellei, E coenophialum, E lolii to control intestinal parasites in livestock.

In addition, the invention provides the use of certain strains of endophyte that have been grown in culture to control intestinal parasites in livestock. These strains can be inoculated under specific stringent conditions into other grass species such as tall fescue and perennial ryegrass and result in the accumulation of loline alkaloids in the leaf, stem and root tissue of these plants.

The endophyte strains Epichloe coenophialum (=Neotyphodium coenophialum), E. occultans, E. siegelli, E. pampeanum, E. uncinatum (new naming Epichloe=Neotyphodium) are preferred.

The invention provides a loline alkaloid selected from the group comprising loline alkaloids in pastures (N-acetyl loline, N-formyl loline, N-acetyl norloline, N-methyl loline) in the treatment of parasitic infestation in livestock, particularly sheep. A range of concentration of loline alkaloid compounds may be useful. Useful concentrations range from 250-25000 μg/g. The concentration selected should be sufficient to deter a number of insect species from feeding on the herbage plants in which they are found.

Any grass species containing loline alkaloids is included within the scope of the invention. Loline alkaloids can be found in endophyte-infected pasture grasses of the following species; Festulolium sp, Lolium perenne, Festuca pratense (syn. Lolium pratense (Huds.) Darbysh., and Schedonorus pratensis (Huds.) P.Beauv.)), Festuca arundinacea (syn., Lolium arundinaceum Darbyshire; Schedonorus phoenix (Scop.) Holub), Lolium multiflorum Lam., Lolium temulentum, Festuca arundinacea, Adenocarpus, Convolulacea, Bromus auleticus and hybrids and/or combinations between these species.

The invention also provides the use of cloned plants, root tissue and seeds of useful endophyte containing plants for use in the treatment of livestock with internal parasitic infections. Loline concentration is dependent on plant genotype and environmental effects but is generally lower in root tissue (up to 2000 μg/g) and higher in seed (up to 25,000 μg/g) than in stems and leaves.

The invention thus provides a method of treating livestock with a composition according to the above described invention.

The invention can provide the following advantages:
Reduced larval mobility, egg hatch, adhesion, embedding, population, faecal egg count (FEC), egg and larval survival in situ, and in faeces, or on plants.
In sheep, cattle, goats, deer, camelids.
Improved live weight gain when lolines are in the diet.
Improved animal performance (ovulation, conception, lambing %, growth rate, carcass weight, increased milk etc.)
Fewer dags and less dagginess in lambs when lolines are present in the diet
Less fly strike in sheep when lolines are present in the diet
Better wool quality
Effect of lolines on pasture of parasite population ie reduced larval and eggs numbers on pasture,
Effect of guttation fluid on egg hatch and L1-L3 larvae numbers,
FEC population,
Faecal decomposition compared to standard endophyte decomposition and effect on egg hatch etc.
Improved animal health
Absence of staggers and heat stress on grazing pastures based on loline producing endophytes.

The invention can be used in a natural form in fresh or preserved rations such as pasture, grain, hay, silage, seed and concentrates.

### BRIEF DESCRIPTION OF THE DRAWINGS

The invention will now be described, with reference to the following drawings and by way of example only in which:

FIG. 1 shows the effect of the pure lolines on the egg hatch of T.circumsincta (A) and Trichostrongylus (B) nematodes in the concentration range 0-1.236 ppm.

FIG. 2 shows graphs A and B in which Trichostrongylus nematodes were hatched in crude loline extract. Graphs D and E show T. circumsincta nematodes hatched in crude loline extract and graphs C and F show Trichostrongylus and T. circumsincta nematodes respectively, hatched in the pure lolines extract

FIG. 3 shows larval migration of T. circumsincta nematodes exposed to lolines and/or loline free grass extract,

FIG. 4 shows larval migration of Trichostrongylus nematodes exposed to lolines and/or loline free grass extracts.

FIG. 5 Larval migration assays with water as the control and diluent. Observe the increase in migration from the control to 6.25% crude lolines extract for both T. circumsincta (graph A) and Trichostrongylus (graph B).

FIG. 6 shows that saline offers greater larval migration than water for both nematode species. The addition of the crude loline extract to distilled water (1% Loline) increased larval migration over 100%.

FIG. 7 shows the adhesion/embedding of T. circumsincta at differing loline concentration. A with crude loline, B-D are using pure lolines. It is a development assay.

FIG. 8 is a final assay for the adhesion/embedding of T. circumsincta.

FIG. 9 shows the weight of lambs grazed on different endophyte pasture types for four weeks in autumn 2013.

### DETAILED DESCRIPTION OF THE INVENTION

The effect of loline alkaloids on intestinal nematodes was determined first using in vitro experiments. The laboratory experiments were aimed specifically at determining the effect of loline alkaloids on the motility and attachment of internal parasites to excised abomasal sections of naïve lambs (reduced nematode motility is a measure of possible treatment effects on nematode life cycle which may translate into reduced faecal egg counts (FEC) in live animals). Reduced FEC can lead to a reduction in pasture contamination consequently slowing down the dynamics of animal infection (Hoste et al., 2006). This assay is an indicator of potential anthelmintic effects or, as a stimulator of the immune exclusion/prompt rejection (IR/PR) response, due to loline alkaloids. Larval migration (a common test to determine the anti-parasitic activity of a chosen compound) was tested. The effects on the nematode egg hatching rate when exposed to lolines was also observed. A reduction in egg hatching may lead to reduced parasite numbers and ultimately reduced parasite burden in the animal. Reduction in any or all of the above parameters was the first step in development of grasses and/or products that could have anthelmintic activity.

Field studies with lambs were undertaken to investigate the potential of loline alkaloids for intestinal parasite control in situ. Loline activity on intestinal nematodes would have benefit to the agricultural industry.

### Method

A number of important aspects of the nematodes' life cycle were studied, In the laboratory we investigated the effect of lolines on the egg hatch and larval migration of the nematodes T. circumsincta and Trichostrongylus colubriformis, and the ability of the nematodes T. circumsincta to adhere to the abomasum in the presence of lolines. Field trials were also carried out to determine the effect of lolines on the nematodes in situ.

### Egg Hatch Assay Methodology

The egg hatch assay is a modification of Dobson et al., (1986).

### Egg Preparation for Egg Hatch Assays

Approximately 100 g of faeces were homogenised using a stomacher. A small amount of water was added to aid in the homogenisation process. The homogenised faeces were poured through a 150 μm mesh sieve which was situated over an empty bucket. The faeces were then thoroughly rinsed with water using a spray hose and the wash through from the sieve was collected into the bucket,

The contents of the bucket were then poured through a 38 μm mesh sieve and the wash through from this sieve was discarded. The matter retained in the sieve was rinsed several times with water and finally using water to wash out the sieve, it was collected into a beaker. This material was poured into 50 ml centrifuge tubes (each tube was only filled to ½). The tubes were then topped up to 50 ml using clean water. Following centrifugation at 1000 rpm for 5 minutes, approximately two-thirds of the water was removed via vacuum suction to waste.

The tubes were then refilled to 50 ml using saturated sodium chloride and centrifuged at 1000 rpm for 5 minutes. Any large floating particulate matter was carefully removed if possible and discarded. Approximately 50% of the supernatant was sucked out using vacuum suction into a storage reservoir. This was transferred into a clean beaker. This step was repeated and the resulting supernatant was combined with the first batch, The eggs were then well mixed and counted using a McMasters slide and a microscope set at 100× magnification to achieve approximately 60 eggs for each well of the assay plate.

The eggs received a final wash in a 25 μm mesh bag, under running water for 10-15 minutes. The volume required to get a minimum of 60 eggs per 50-100 μl was calculated and the eggs were collected into a new 50 ml tube and the volume checked to ensure the eggs had not been over diluted during collection. The eggs were then well mixed and a subsample was counted to ensure the presence of the required number of eggs (the mixture can be diluted or concentrated as required).

### Egg Hatch Assay

The nematode eggs for the egg hatch assay were collected as above. 50-100 μl of eggs (aim for >50 eggs) were added to 1.5 ml of the desired loline solution per well in a clean, 48 well plate. The treatments including a distilled water control were run in triplicate on the plates. The plates were then incubated at ˜28° C. for 2-4 days. After incubation the number of larvae and unhatched eggs were counted under a microscope. The % hatched was calculated using the following formula:

% hatched=(Number larvae/(Number larvae+Number eggs))*100

The loline solutions were diluted using distilled water. It was established in previous experimentation that distilled water was the best control and diluent due to the high egg hatch percentage in 100% distilled water (Appendix 1).

Initially a crude extract of lolines was used (the extract contained a large amount of plant material). Once a reaction area was established a loline mixture of higher purity (containing NFL, NAL, NML, NANL and underived loline (UDL)) was used. The final assays for T. circumsincta and Trichostrongylus were run using the following dilutions of lolines: 0 ppm (control), 206 ppm, 412 ppm, 618 ppm, 824 ppm, 1,030 ppm and 1,236 ppm.

### Larval Migration Assay Methodology

The larval migration assay is a modification of Wagland et al, 1992.

T circumsincta or Trichostrongylus larvae were exsheathed by adding >80 μl of bleach (Milton's fluid) per 1 ml of larval suspension and mixed by hand rotation. The larvae were observed under an inverted microscope at 100× magnification and when at least ˜90-95% of larvae had exsheathed they were washed in tap water and centrifuged three times to remove excess bleach (small bursts of spinning were used to try and avoid the larvae clumping together). In case of clumps, the larvae were then re-suspended by drawing the solution into a syringe through a fine gauge needle (minus the bevelled edge) and then forcefully expelling them back into the tube. This was repeated until they were unclumped.

A 25 μl subsample of larvae was counted and it was ensured that 100 μl to a maximum of 300 μl contained at least 200 larvae as ˜100 larvae were needed per sample and the assay was run on the plate in duplicate. Approximately 200 larvae (100-300 μl of larval suspension) were put into each treatment tube (one tube per treatment) along with 2 ml of the appropriate concentration of lolines/extract or control. The control and diluent were 0.85% saline i.e. physiological strength; it was established in previous assays that water significantly reduced the larval migration when compared with migration percentages using physiological strength saline (see Appendix I for data).

The tubes were then incubated at ˜37° C. for 2 hrs.

One ml of treatment solution was added to each well of a clean 24 well plate. A maximum of 6 treatments could be run as there were 2 wells per treatment (duplicate) and each sample well required an empty well for later i.e. 12 wells with solutions and 12 wells empty. Clean inserts made of tubing with 25 μm mesh wrapped tightly over and around the base (held in place by a larger piece of tubing sized to be a tight fit into the wells on the plate), were placed into each well containing solution. The inserts were pushed nearly all of the way to the bottom of the well but a small gap i.e. 2-3 mm was left between the bottom of the well and the bottom of the insert. It was ensured that there was no air present between the mesh and solutions in the well by squeezing up a small amount of solution into the inserts which in turn removed any trapped air.

Following the 2 hr incubation the tubes were gently centrifuged and about 1.3-1.5 ml of solution was removed. The larvae were then re-suspended as earlier and half was put into each of the two inserts containing that treatment This was done by gently pipetting down the inside of the insert tube (aim of ˜100 larvae per well). This was repeated for all treatments. The plate was then placed in the 37° C. incubator again for 2 hrs.

After incubation, the following was done one by one. An insert was carefully removed and with a transfer pipette and a little water. The outside of the insert was gently rinsed into the well to remove any larvae adhering to the underside of the mesh. The inside of the insert was carefully rinsed into a corresponding empty well to remove any larvae in the inside of the insert This was repeated for all treatments.

The larvae in each well were counted under an inverted microscope at a suitable magnification, Helminthological iodine was added to each well to kill the larvae for easier counting. Finally, the number of larvae in the original well was compared to the number of larvae that were rinsed out of the inside of the insert. The larvae that moved through the mesh into the well had migrated. The larvae that remained inside the insert had not migrated. The percent migration was calculated as below:

% Migration=(Number larvae travelled through the mesh/(Number larvae travelled through the mesh+Number of larvae retained in the insert))*100

A crude lolines extract was initially used in concentrations of 100% (approximately 23,000-31,000 ppm of lolines), 50%, 25%, 12.5%, 6.25% and 0%. The crude extract was lolines with a substantial amount of plant material. This extract significantly reduced the larval migration in both the T. circumsincta and Trichostrongylus assays. Next we used the pure lolines solution at the same % levels (ranging from ˜13,000-25,000 ppm for T.circumsincta and ˜13,000-72,000 ppm for Trichostrongylus of lolines depending on which pure preparation) was used. Overall the pure loline had little if no effect on the migration of either species of nematode. Next we tried a crude extract with no loline i.e. just the plant material to see if it was the plant material that had effected the larval migration in the crude lolines extract. This nil lolines extract also had no effect on the nematodes. Finally we tried mixing the pure lolines (different pure lolines mix depending on timing of when the assays were carried out; see above for details) with the nil lolines crude extract at a ratio of 1:1 and tested these with both T. circumsincta and Trichostrongylus at a 50%, 25%, 12.5%, 6.25% and 0% lolines (50% lolines equals an undiluted 1:1 mix). This was to mimic the crude lolines extract that had effected larval migration. The majority of the work was carried out using T. circumsincta, however at least one of each type of assay performed was carried out with Trichostrongylus to ensure that both nematode species behaved in a similar way.

### Larval Adhering/Embedding Assay

This method is a minor modification of the Lincoln University ‘Explant Tissue using Ostertagia larvae (Teladorsagia circumcincta)’ Standard Operating Procedure” which was based around in vitro methods in studies on explant culture of human colon and by Rosberg et al., (1991) in Helicobacter adherence studies using pig gastric mucosa.

### Abomasum Collection (Post-Mortem)

The abomasum was removed immediately from a naive lamb following ethical euthanasia, it was then rinsed gently in warm physiological saline (0.85%) to remove most of the adherent mucous layer.

### Larvae Exsheathment

T. circumcincta larvae were exsheathed by adding 80 μl of 5% hypochlorite solution per 1 ml of larval suspension in a 15 ml centrifuge tube. Larvae were mixed thoroughly and left for 2 mins. When 99 % of larvae had exsheathed larvae were washed to remove excess hypochlorite. Larvae were washed and centrifuged 3 times for 5 minutes at 2000 rpm. Exsheathed larvae were resuspended in warm saline. Larval concentration was counted and adjusted to 500-1000 larvae per 100 μl.

### Adhering/Embedding Assay

Abomasal sections of approximately 2×2 centimetres were cut from the folds in the abomasum using a scalpel and/or scissors. Each section was placed into a well of a Corning 6 well plate and gently flattened out using forceps. Warm Hanks medium with Hepes (Gibco-Life Technologies) was pipetted around the tissue sections to surround but not immerse them. Modified 5 ml syringe barrels (lower end removed and O-rings added to keep them in place when the plate lid was added), were placed in the centre of each piece of tissue, thus providing an isolated cylinder for the larvae. The lid (which was also modified by having holes drilled to neatly fit over the syringe barrels and O-rings), was placed firmly onto the plate, it was secured with rubber bands to provide an effective seal between the syringe barrel/O-rings and tissue. 1 ml of treatment or control solution (physiological saline) was added into each syringe barrel to which a 100 μl of exsheathed larvae was added. Each assay was run in triplicate with treatment and controls.

Assay plates were transferred to a modified Tupperware container and the lid was sealed. This container was then flooded with pure oxygen for approximately 30 seconds. Each container had a hole drilled into the lid and plugs were inserted. The plates were then placed in an incubator at 38° C. for 3 hours. The aim was to restrict the time taken from slaughter of the lamb to the start of the incubation to be no more than 20 minutes.

After incubation tissue samples were removed and subjected to a vigorous wash procedure. Each tissue section was washed by dunking them at least 30 times in 25 ml of physiological saline in an appropriately labelled 50 ml centrifuge tube. After washing, tissue samples were then placed into a second centrifuge tube (‘digest’) and digested in 50 ml of 1% pepsin 1% HCl solution at 38° C. overnight. Syringe barrels and wells were thoroughly rinsed with physiological saline and this was collected into the same 50 ml centrifuge tube and then made up to 50 ml using physiological saline (‘wash’).

After mixing, 2×1 ml sub samples were taken per tube and larvae counted under a microscope. The number of larvae for each sample was taken as the average of the two counts. The percentage of larvae embedded in the tissue determined by the following formulae.

% embedded=(No. of Larvae in Digest/(No. of Larvae in Digest+No. of Larvae in Rinse)) *100

The crude lolines extract (˜31,000 ppm) was used initially and then a pure lolines extract (approximately 72,000 ppm) at concentrations of 0 (control of physiological saline 0.85%), 6.25%, 12.5%, 25%, 50% and 100%.

### Field Trials

### Safety of Lambs and the Effect on Intestinal Parasites of Lambs, Grazed on Grasses Containing the U2 Endophyte (Which Produces Loline Alkaloids)

The twin objectives of these experiments were to check safety of lambs and to observe population changes of intestinal parasites in lambs grazing pastures containing loline alkaloids.

Pastures were prepared by hard grazing by ewes, fertilizer, mowing, and irrigation as necessary.

In mid-November, 15 weaned Iambs were drenched and grazed on 3×0.1 Ha blocks sown with the 3 grasses (Barrier U2, Matrix SE and Matrix LE). The lambs were inoculated twice, 7 and 10 days later, with L3 larvae. Fifteen lambs were rotationally grazed on one block of each grass line for one week. The following week the same lambs moved on to another block of the same grass line for one week, and so on through subsequent weeks. Faecal egg counts were taken at 0, 10, 21, 28 and 35 days after grazing started. Fasted lamb weights were taken at the commencement and end of the experiment and weekly during the experiment.

Faecal samples were collected from the lambs for faecal egg count (FEC).

Dags of each lamb were scored (1-5 max) for the amount of soiling (faecal contamination) around the anus of the lamb. The firmness of the stool was scored on a 1-5 basis (1-firm marble, 5-very watery i.e. no stool).

### Results

### Egg Hatch

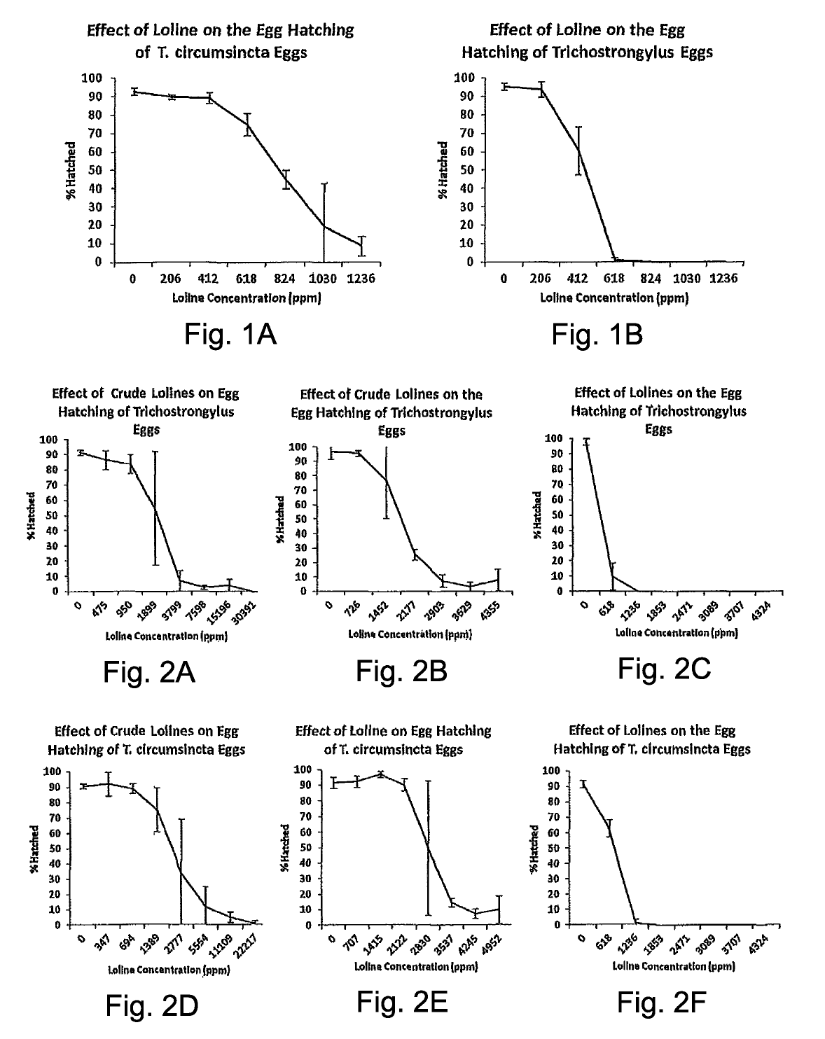

The crude loline extracts showed a significant reduction in egg hatch levels for T. circumsincta and Trichostrongylus at about 3,000-4,000 ppm of lolines. Focus was made on the range of 0-4,324 ppm using the pure loline preparation. When the pure lolines preparation was used, there was little egg hatching occurring at concentrations of 1,236 ppm. Egg hatch results for both nematode species at a range of 0-1,236 ppm of lolines are shown in FIG. 1.

At 1,236 ppm egg hatch of T. circumsincta was 8.8%. See FIG. 2 for other egg hatch assays. Egg hatch of Trichostrongylus was more sensitive to loline deterrence than T. circumsincta. Egg hatch of Trichostrongylus was 0.8% at 618 ppm loline and zero at 824 ppm.

### Additional Assay Information

Egg hatch

TABLE 1
Egg hatch data showing that water allows a much greater egg hatch percentage than
physiological saline. This means that water was a much better diluent and control than the saline.

| | | Replicate | | | | | | | | | |
|---|---|---|---|---|---|---|---|---|---|---|---|
| | | 1 | 2 | 3 | 4 | 5 | 6 | 7 | 8 | MEAN | SD |
| SALINE | Larvae | 33 | 22 | 26 | 45 | 46 | 38 | 25 | 33 | | |
| | Eggs | 14 | 19 | 12 | 7 | 17 | 16 | 17 | 15 | | |
| | % Hatched | 70.2 | 53.7 | 68.4 | 86.5 | 73.0 | 70.4 | 59.5 | 68.8 | 68.8 | 9.7 |
| H₂0 | Larvae | 55 | 42 | 57 | 44 | 54 | 50 | 38 | 58 | | |
| | Eggs | 0 | 3 | 4 | 3 | 0 | 1 | 2 | 2 | | |
| | % Hatched | 100.0 | 93.3 | 93.4 | 93.6 | 100.0 | 98.0 | 95.0 | 963 | 96.3 | 2.8 |

### Larval Migration

T. circumsincta

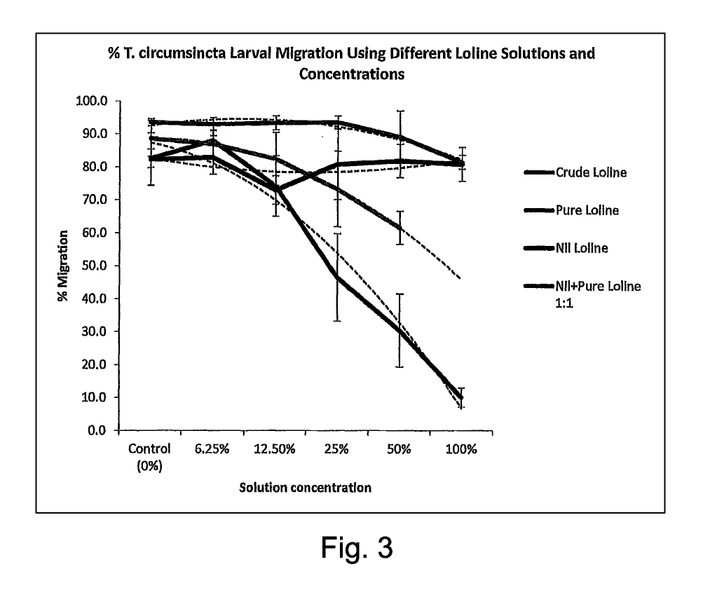

The crude extract had a significant effect on larval migration. This is shown in FIG. 3. The pure loline solution had little if no effect on the migration of the T. circumsincta larvae. It was thought that maybe some other component of the crude extract was having the inhibitory effect on larval migration and hence the experimentation with the nil loline crude extract. The nil loline extract also had no effect on the T. circumsincta larval migration. The proposition that loline was working in conjunction with another component or components was tested and that they were both/all are required to have an inhibitory effect on larval migration. When the pure loline solution was mixed with the nil loline crude extract at a ratio of 1:1, the mixture partially inhibited the larval migration of the T. circumsincta nematodes showing that loline alkaloids reduce migration of T. circumsincta larvae. The trend lines FIG. 3 (2nd order polynomial was used as ‘best fit’), indicate that the 1:1 mix of nil crude extract and pure loline has a very similar effect to that of the crude lolines extract.

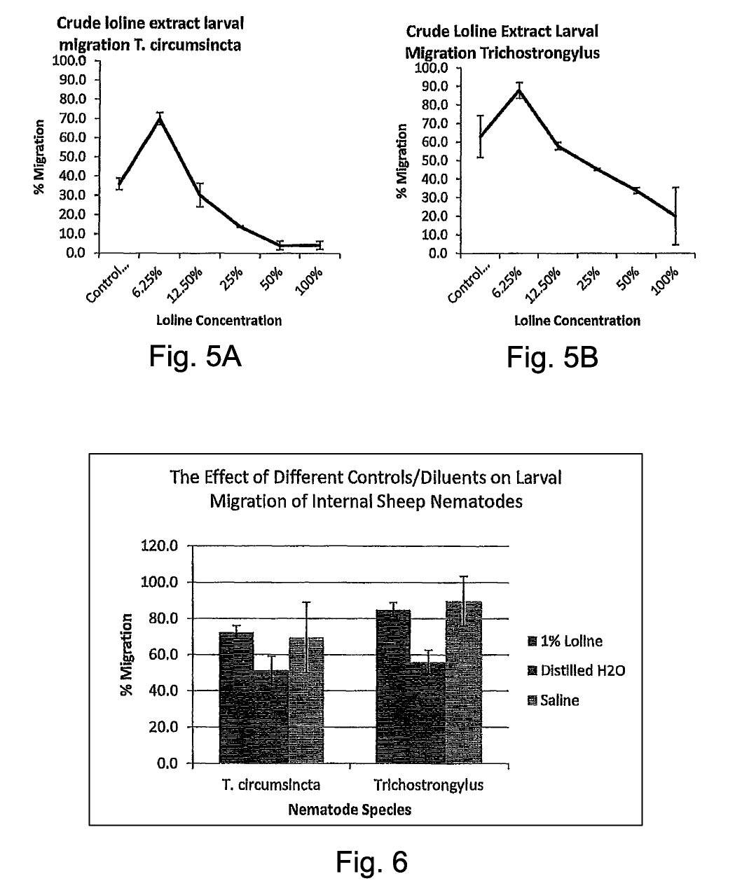

The results presented for T. circumsincta larval migration are averages of three assays except for the crude extract which is from one assay. Previous work in the early stages of experimentation showed that crude lolines had a major impact on the larval migration of the T. circumsincta nematodes. These results are presented in FIG. 5. Contrary to normal practice water (versus saline) was initially used as the control and diluent. Greater migration was observed using the physiological saline solution (FIG. 6).

### Trichostrongylus

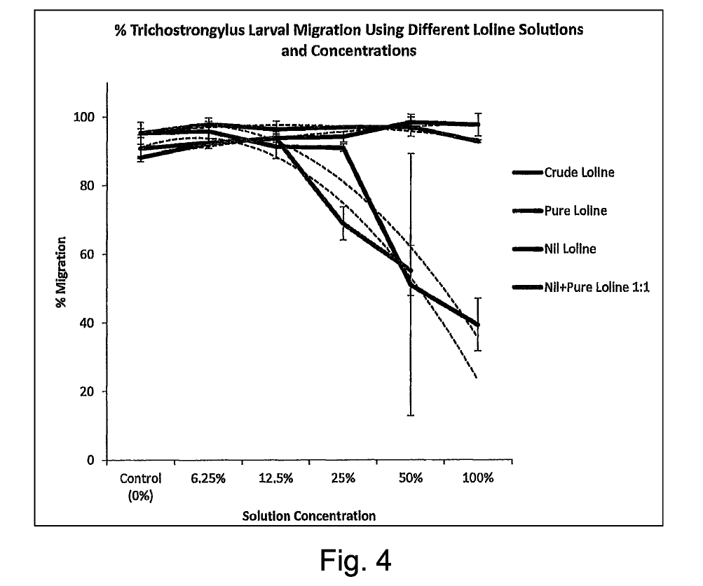

The larval migration of Trichostrongylus (FIG. 4) was very similar to the T. circumsincta migration i.e. loline on its own and crude nil extract had little effect on larval migration but the combination of pure loline and nil extract reduced larval migration similarly to the crude loline extract.

The trend lines in FIG. 4 (2nd order polynomial were used as ‘best fit’), indicate that the 1:1 mix of nil crude extract and pure loline has a very similar effect to that of the crude loline extract.

The results in FIGS. 3 and 4 are from assays for each species. Experimentation showed that crude lolines had a major impact on the larval migration of the Trichostrongylus nematodes, Contrary to normal practice water (versus physiological saline concentrations) was initially used as the control and diluent. Greater migration was observed using the saline solution (FIG. 6).

It was observed that the % migration of the control for the larval migration assays was usually significantly below the % migration of the first crude lolines extract dilution (FIG. 6), It was clear that the loline extract was having any inhibitory effect on the larval migration at high concentrations but at a low concentration it was promoting migration when compared with the control which was water. It was subsequently found that water did indeed partially inhibit migration when compared with saline (FIG. 6). Consequently 0.85% saline was used as the control and diluent.

The graph shown in FIG. 6 shows that larvae of both T cicumsincta and Trichostrongylus in saline solution migrate more than in water. FIG. 6 also demonstrates that the addition of the crude loline extract to distilled water (i.e. 1% loline) increased larval migration over the 100% distilled H₂O.

### Adhering/Embedding Assay

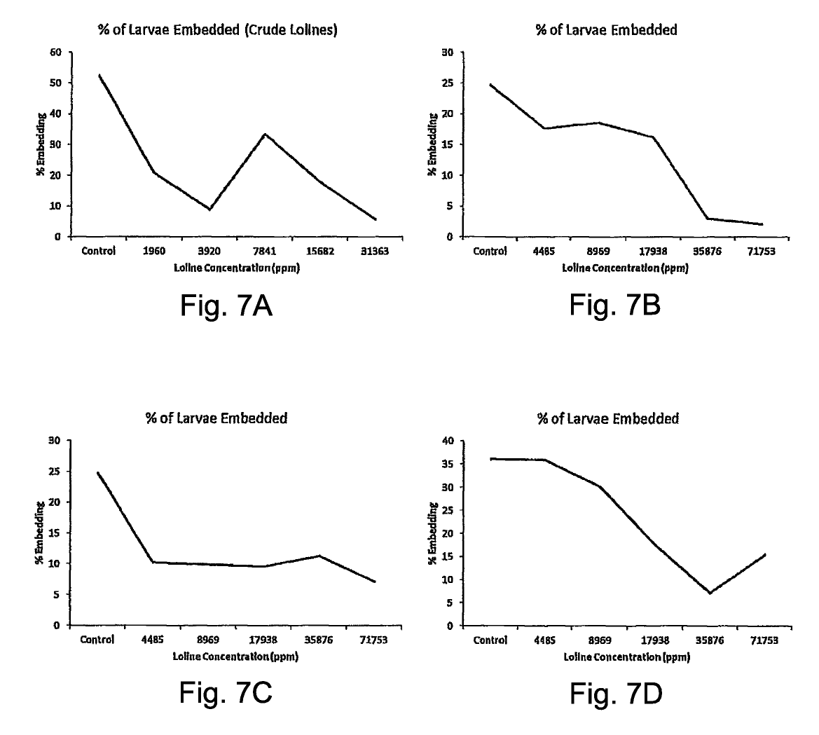

Loline alkaloids have an effect on larval adhesion/embedding in abomasal wall tissue. This is shown in FIG. 7. The crude lolines extract reduced larvae adhering to the abomasum in the presence of lolines; the same effect was demonstrated in the presence of pure lolines.

Pure lolines at 4,500 ppm had a major impact on adhesion/embedding of T. circumsincta to the abomasum. FIG. 7(D) shows that the loline effect starts at 9000 ppm. The crude lolines at 6.25% reduced the number of larvae embedding and this equates to about 1,900-2,000 ppm of lolines having an effect. There are couple of anomalies (the 25% in the crude lolines and the 100% in Graph D), but the overall trend is very clear.

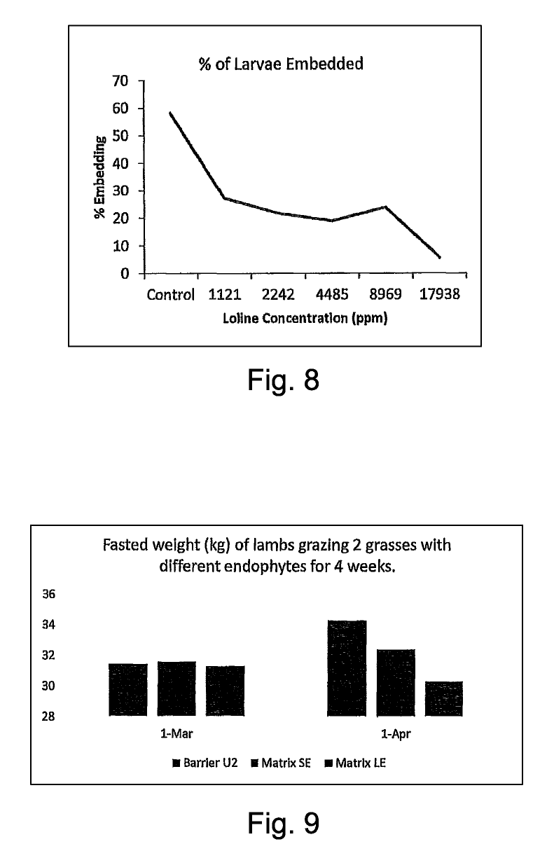

Using the finalised method, the results showed exactly the same trend as the method development assays. However, the concentration of lolines required for a significant reduction in larval adherence to the abomasum were greatly reduced compared to the concentration of lolines in the initial stages of method development (FIG. 8).

At 1,120 ppm (1.56%) of lolines there is a 30% reduction in T. circumsincta larval adhesion/embedding in the abomasum tissue. The level of embedding decreases to 6% of larvae embedding at 18,000 ppm (25% lolines).

### Grazing Experiments

Endophyte contained within the grass influenced and both lamb live weight gain, dag score, and faecal egg count (FEC) (FIG. 9 and Table 2). Lambs grazing on the Barrier U2 had less dags and a firmer stool (i.e. lower moisture content), had lower FEC, and the lambs put on more weight than those grazing the other two endophyte grass combinations.

TABLE 2
Dag score, faecal egg count (FEC) of lambs after grazing two grasses
with different endophytes for four weeks Mar. 8-Apr. 5, 2013.

| | | Mean | Mean | Mean | | |
|---|---|---|---|---|---|---|
| | Dag Score | stool | dag | wt. | FEC | |
| | of 1 | firmness | score | gain | total | No >1000* |
| Barrier U2 | 10 | 2.54 | 1.33 | 2.86 | 1538 | 5 |
| Matrix SE | 1 | 3.36 | 3.17 | 0.8 | 2353 | 11 |
| Matrix LE | 2 | 3.27 | 3.67 | −2.0 | 3801 | 9 |

*Number of lambs with faecal egg counts greater than 1000.

The number of lambs with dag score of #1 shows a large difference between the lambs grazing Barrier U2 and lambs grazing both of the other grasses. These numbers are reflected in the firmness of stool score, average dag score, and FEC.

TABLE 3
Dag score, stool firmness score, weight (wt.) gain and
faecal egg count (FEC) of lambs after grazing two grasses
with different endophytes for four weeks Mar.-Apr. 16, 2014.

| | | | | | |
|---|---|---|---|---|---|
| Dag | Mean | Mean | Mean | | No. lambs |
| Score | stool | dag | weight | Ave | with |
| of 1 | firmness | score | gain (g) | FEC | FEC >1000 |

| | | | | | | |
|---|---|---|---|---|---|---|
| Barrier U2 | 1 | 2.5 | 2.1 | 400 | 758 | 2 |
| Matrix SE | 0 | 2.8 | 2.9 | 200 | 681 | 0 |
| Barrier LE | 4 | 2.1 | 1.9 | −600 | 1036 | 7 |

TABLE 4
Dag score, stool firmness score, and faecal egg count (FEC)
of lambs after grazing three grasses with different endophytes
for six weeks, Apr. 16-May 28, 2014.

| | Dag | Mean | Mean | Mean | | No. lambs |
|---|---|---|---|---|---|---|
| | Score | stool | dag | wt. | Ave | with |
| | of 1 | firmness | score | gain (kg) | FEC | FEC >1000 |
| Barrier U2 | 2 | 2.73 | 2.25 | 3.8 | 682 | 2 |
| Matrix SE | 0 | 3.1 | 3 | 2.9 | 1822 | 6 |
| Barrier LE | 0 | 2.58 | 2.08 | 3.5 | 873 | 4 |

The alkaloid concentration was determined in herbage samples taken from each plot immediately prior to grazing each week. Herbage samples were analysed to determine the alkaloid profile of each grass genotype.

TABLE 5
Alkaloid profiles of grazed plots.

| | | | Ave. |
|---|---|---|---|
| | | | Concentration |
| Alkaloid | Sample source | Date | μg/g |
| Peramine* | Plots 1, 6, 7, 11 | Feburary, April/May | 4.8, 3.6 |
| Lolines* | Plots 2, 4, 8, 10 | Feburary, April/May | 1817, 2203, |
| Ergovaline** | Plots 1, 2, 3 | April | 4, 0, 0 |

*Peramine and loline measured by Cropmark Seeds.
**Ergovaline by Wade Mace, AgResearch, Palmerston North.

None of the above alkaloids were detected in samples taken from plots other than those nominated.

### Dry Matter Production

In the 2013 experiment and the April to May 2014 experiment, dry matter available prior to grazing was estimated by pasture probe. In the 2014 experiment residuals after grazing were also measured. From these numbers an estimation of dry matter consumption was derived. These numbers indicated little difference in pasture consumption by the lambs between the different grass×endophyte treatments suggesting other influences on lamb live weight gain/loss.

### Grazing Trial 2015

### Methods

Four areas (0.4 Ha each) of 3 grass varieties (Barrier U2, Barrier Low endophyte and Matrix Standard endophyte (i.e. 12 blocks in total) were sown in the spring 2014 at Sharplins Road, Aylesbury, Canterbury. After grazing by ewes and lambs, 10 lambs were set stocked on each block in March 2015 for 62 days. The lambs were weighed into groups that were randomly allocated to treatments. Extra lambs were added as required to different blocks to ensure similar offer to each lamb. Lambs were weighed and dry matter assessed by electronic pasture probe each week. Faecal grab samples were taken on 6 occasions over the experimental period for faecal egg count assessment by Gribble Veterinary Pathology. Scoring (1-5) of faecal contamination around the anus of each lamb (Dag Score) was taken at each weighing. Dags were removed and weighed after 47 days. A faecal grab sample was taken on one occasion for dry matter determination.

Grass samples were taken for alkaloid analysis and a separate grass samples were taken on two occasions for assessment of L3 grass larval population at the end of the experiment. Samples taken for alkaloid analysis were frozen as soon as possible after harvest and held at −20° C. for freeze drying and grinding before extraction and analysis.

### Results

One lamb was removed from the experiment due to flystrike before assessments commenced. Another lamb was observed to suffer from mild perennial ryegrass staggers on 2 occasions when being mustered for the weekly weighing. Both these lambs were grazing the Matrix SE treatment. Both of these lambs fully recovered.

### Dry Matter Assessments

The dry matter production over the period of the experiment is presented in Table 6. The Matrix SE plots had higher covers at the start of the experiment and retained this advantage throughout the experimental period. Lamb numbers were adjusted to maintain the offer to each lamb as uniform as possible.

TABLE 6

| | Average dry matter assessment of each treatment at the | | |
|---|---|---|---|
| | start and end of the experiment | | |
| | Matrix SE | Barrier LE | Barrier U2 |
| 23 Mar | 2304 | 1842 | 1427 |
| 7 May | 1906 | 1699 | 1504 |

### Lamb

Average lamb weight increased over the over the grazing period from an initial weight of 33 kg LW to between 35 kg and 41 kg (Table 10). Lambs grazing Matrix SE averaged 35.6 kg at day 62. Lambs grazing Barrier U2 averaged 39.9 Kg at the end of the experimental period and those grazing Barrier LE 40.3 Kg. There was no difference in the rate of weight gain between lambs grazing on each of the Barrier treatments (112-118 g/day) but weight gain on Matrix SE was significantly different (41 g/day, p<0.001) to both Barrier treatments.

Average dag scores (1-5) for the lambs were different between treatments (p<0.05) (Table 8). Lambs grazing Matrix SE always had the highest score (i.e. most dags). Lambs grazing Barrier U2 always had the lowest score which reached significance at assessment on Day 34 (p<0.001) and Day 62 (p<0.05).

Dags were removed and weighed on Day 47 and large differences in average dag weight between treatments were recorded and largely reflected the dag score reported above. Dag weight of lambs grazing Barrier U2 averaged 50 g, those on Barrier LE averaged 150 g and those grazing Matrix SE averaged 310 g (p<0.05). Average faecal egg count increased in all treatments over the grazing period to an average over all the lambs of 1020/g (averaged over the last 3 collection date i.e. day 34, 55 and 62) (Table 9). There was a significant difference in average FEC between lambs grazing BU2 and Matrix SE (p<0.05) when averaged over the last 3 collection dates). The FEC of lambs grazing BU2 were consistently lower than those grazing Barrier LE but the differences failed to reach significance (p<0.05).

### Alkaloid/Endophyte Testing

The concentration of peramine, ergovaline, and loline (averaged over 4 replications) in the three grasses during the experiment are shown in Table 7. The qualitative results are as expected i.e. the two endophytes involved resulted in the presence of the anticipated alkaloids. There was no evidence of any alkaloids in the Barrier LE treatment. This result was supported by immunoblot tests for the presence of endophyte, Ergovaline and peramine were found in the Matrix SE plots only. Loline alkaloids were found only in the Barrier U2 plots.

TABLE 7
Alkaloid concentration in grass samples (average over 4 replications)
taken from each plot at the midpoint of the experiment*

| | Average alkaloid concentration (μg/g) in grass samples | | |
|---|---|---|---|
| | Loline | Ergovaline | Peramine |
| Barrier U2 | 12,309 | nil | nil |
| Barrier LE | nil | nil | nil |
| Matrix SE | nil | 0.74 | 18.9 |

*All alkaloid analyses were undertaken in the laboratories of Cropmark Seeds Ltd.

### Field Larval Counts

TABLE 8
Mean dag scores and dag weight of lambs grazing three grasses
containing different endophytes over 62 days

| | Dag scores at day number after March 4 | | | | | | |
|---|---|---|---|---|---|---|---|
| | (e.g., 20 = March 24) | | | | | | |
| | Dag | Dag | Dag | Dag | Dag | Dag | Dag wt (kg) |
| Treatment | [20] | [26] | [34] | [40] | [47] | [62] | on day 47 |
| BU2 | 1.45 | 1.40 | 1.62 | 1.70 | 1.67 | 1.43 | 0.05 |
| BLE | 1.95 | 2.20 | 2.35 | 2.32 | 2.32 | 1.98 | 0.15 |
| MSE | 3.37 | 3.65 | 3.72 | 3.57 | 3.27 | 2.36 | 0.31 |
| LSD (5%) | 0.58 | 0.69 | 0.29 | 0.80 | 0.89 | 0.55 | 0.19 |
| LSD (1%) | 0.87 | 1.04 | 0.44 | 1.21 | 1.35 | 0.84 | 0.29 |

TABLE 9
Mean faecal egg count (FEC) of lambs grazing three grasses
containing different endophytes over 62 days

| | Faecal egg counts (eggs/gram) | | | | | | |
|---|---|---|---|---|---|---|---|
| | on day number after March 4 | | | | | | |
| | (e.g., 19 = March 23) | | | | | | Average FEC |
| | FEC | FEC | FEC | FEC | FEC | FEC | over last |
| Treatment | [5] | [12] | [19] | [34] | [55] | [62] | three dates |
| BU2 | 132 | 399 | 282 | 882 | 810 | 820 | 837 |
| BLE | 150 | 379 | 241 | 1143 | 1193 | 833 | 1056 |
| MSE | 208 | 472 | 221 | 1113 | 1320 | 1097 | 1177 |
| LSD (5%) | 99 | 213 | 151 | 266 | 440 | 424 | 336 |
| LSD (1%) | 150 | 323 | 229 | 403 | 666 | 643 | 509 |

TABLE 10
Mean fasted lamb weights of lambs grazing three grasses
containing different endophytes over 62 days

| | Fasted lamb weights on day number after March 4 | | | | | | | |
|---|---|---|---|---|---|---|---|---|
| | (e.g., 20 = March 24) | | | | | | | Total |
| | Wt. | Wt. | Wt. | Wt. | Wt. | Wt. | Wt. | weight |
| Treatment | [0] | [20] | [26] | [40] | [47] | [54] | [62] | gain |
| BU2 | 33.0 | 35.4 | 37.7 | 39.4 | 38.4 | 39.8 | 39.9 | 6.9 |
| BLE | 33.0 | 35.2 | 38.2 | 39.7 | 39.0 | 40.0 | 40.3 | 7.3 |
| MSE | 33.0 | 34.0 | 36.3 | 36.1 | 35.6 | 35.5 | 35.6 | 2.6 |
| LSD (5%) | 0.1 | 0.7 | 0.8 | 0.7 | 0.9 | 0.6 | 0.7 | 0.7 |
| LSD (1%) | 0.1 | 1.0 | 1.2 | 1.1 | 1.3 | 0.9 | 1.1 | 1.1 |

TABLE 11
Mean pasture larval counts/kg DM from samples taken on two
separate occasions after grazing by lambs for 62 Days
Average pasture larval counts/Kg DM

| | 6 May | 26 May |
|---|---|---|
| Barrier U2 | 7240 | 3255 |
| Barrier nil | 3740 | 2269 |
| Matrix SE | 6720 | 9266 |
| Mean | 5900 | 4930 |

At first count (6 May) after grazing pasture larval counts did not differ between the grass treatments (Table 11). Mean pasture larval count decreased from May 6 to May 26, After 3 weeks (26 May) pasture larval count was less on the Barrier treatments than from Matrix SE and larval counts on the Barrier U2 treatment are 50% lower than the first count (May 6).

### Discussion

There is clear evidence from this experiment that lambs grazing Matrix SE produced more dags, had higher FEC, and gained less weight over the experimental period, than lambs grazing Barrier with or without U2 endophyte. In the present experiment the weight gain of lambs grazing Barrier LE and Barrier U2 were not different but there was a significant difference in dag score, dag weight and a non-significant difference in FEC in lambs grazing these two grasses.

Faecal egg count almost certainly underestimates the worm burden in badly infected lambs due to dilution in extremely loose stools and the inability to satisfactorily obtain a representative sample of those faeces. This will have been a factor in the results indicated in Table 9.

The host grass on which larvae are found after deposition in faeces may effect larval survival, as indicated by the greater decline in larval counts on Barrier U2 pastures than the other treatments (Table 11). This could have significance to reinfection of lambs grazing such pastures and the ultimate worm burden the ingesting animal. These results suggest a practical difference between Barrier LE and Barrier U2 in the effect of the endophyte on intestinal parasites and support field observations of lambs grazing pastures containing U2 infected grasses. These results are supported by laboratory assays which indicate a significant effect of loline containing endophytes on egg survival, larval motility and adhesion in the abomasum.

### Summary of Field Experiments

Lambs grazing the Festulolium grass cv. Barrier containing the U2 endophyte in autumn clearly sustained a lower FEC in the three years of data presented which may indicate a lower worm burden than lambs grazing the other grasses. The lower FEC was reflected in higher weight gains of the lambs grazing Barrier U2 in 2 of the 3 years. The FEC of lambs grazing Matrix SE in 2014 is probably a significant under estimate of the worm burden in those lambs as it was not possible to collect faecal samples from 3 lambs due to the looseness of the stool.

### Conclusions

The results of these experiments and assays overall show that loline alkaloids reduce egg hatch, and larval motility of Teladorsagia circumsincta and Trichostrongylus colubriformis, and embedding of larvae of T. circumsincta in naïve lamb abomasal sections. Grazing studies with lambs have shown that loline containing pasture swards reduced FEC and dags compared to lambs grazing similar pasture swards that do not contain loline alkaloids. These lambs also showed improved live weight gain in the presence of loline alkaloids in the sward in 2 of the 3 years of the experiments compared to similar swards devoid of loline alkaloids. In the third year of the experiments there was no difference in lamb live weight gain between the nil and U2 Barrier treatment but a significant difference between both those treatments and lambs grazing on Matrix SE.

Although the invention has been described with reference to specific embodiments, it will be appreciated by those in the art that variations and modifications may be made to those embodiments without departing from the scope of the invention as described in this specification.

For example, although the experimental data has been carried out on sheep, the invention may also be applied to other livestock including cattle.

Although only certain species of meadow fescue have been described the invention also applies to any fodder species suitable as a crop for livestock.

### INDUSTRIAL APPLICABILITY

The invention will be useful in the livestock industry. The use of loline alkaloids in grasses consumed by livestock will assist in reducing the rates of infection of livestock by internal parasitic infections. The alkaloids will reduce the infection rate in the body of animals consuming the grass and hence the health and performance of the livestock will be improved. This in turn will lead to an improvement in the economic return to farmers.

### REFERENCES

Hoste, H., Jackson, F., Athanasiadou, S., Thamsbourg, S. M., & Hoskin, S. O. (2006). The effects of tannin-rich plants on parasitic nematodes in ruminants. Trends in Parasitology 22; 253-26;

Wagland, B. M., Jones, W. O., Hribar, L., Bendixsen, T., & Emery D. L. (1992). A new simplified assay for larval migration inhibition, International Journal for Parasitology 8:1183-1185

1. A loline alkaloid for use in the control of internal parasitic infestation in livestock,

2. The loline alkaloid for use according to claim 1 in which the livestock are ruminants selected from the group consists of goats, cattle and sheep.

3. The loline alkaloid for use according to claim 2 in which the livestock are sheep.

4. The loline alkaloid for use according to claim 1 in which the internal parasites controlled are nematodes.

5. The loline alkaloid for use according to claim 4 in which the internal parasites are intestinal nematodes.

6. The loline alkaloid for use according to claim 5 in which the parasites treated include: abomasum worms, duodenum worms, large intestine worms and small intestine worms.

7. The loline alkaloid for use according to claim 6 in which the intestinal parasites include Teladorsagia circumsincta and Trichostrongylus colubriformis.

8. The loline alkaloid for use according to claim 1 in which the loline alkaloid includes any compound with the following chemical structure with R′ and R″ denoting variable substituents that can include methyl, formyl and acetyl groups giving rise to different forms of loline including N-formylloline (NFL); N-acetyllolinel (NAL); N-methylloline (NML); and N-acetyl norloline (NANL)

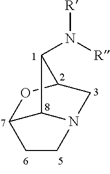

9. The loline alkaloid for use according to claim 8 in which the loline alkaloid is selected from the group contesting of: NFL (N-Formylloling, NAL (N-acetylloline) NANL (N-acetyl norloloine) and NML (N-methylloline).

10. A grass species for use in the control/treatment of internal parasitic infestation in livestock in which a loline alkaloid according to claim 8 in the grass to limit the population and provide control of intestinal parasites in ruminants.

11. A plant species containing a loline alkaloid, for use in the treatment and/or control of internal parasitic infestation in sheep.

12. The plant species for use according to claim 11 in which the plant species is selected from the group comprising: Lolium perenne, Lolium multiflorum, Lolium temulentum, Festuca arundinacea, Festuca pratensis, and any hybrid combinations of these species, Bromus auleticus, Adenocarpus species, Argyreia mollis.

13. The plant species for use according to claim 12 which is a cereal or forage containing loline alkaloids, or any endophyte x grass combination including the endophytes Epichloe coenophialum (=Neotyphodium coenophialum), E. occultans, E. siegelli, E. pampeanum, E. uncinatum (new naming Epichloe=Neotyphodium) producing lolines in forage roots and seed, for use in the treatment/control of intestinal parasites in ruminants.

14. A plant extract containing a loline alkaloids for use in
the treatment of livestock for internal parasitic infestation.

15. Plant containing a plant extract as claimed in claim 14 for use in the treatment of livestock for internal parasitic infestation.

16. An endophyte selected from the group comprising: Epichloe uncinata (syn. Neotyphodium uncinatum), Epichloe spp. (syn. Neotyphodium species), E. seigellei, E. coenophialum, E. lolli for use in the treatment of livestock for internal parasitic infestation.

17. Endophyte strain selected from Epichloe coenophialum (=Neotyphodium coenophialum), E. occultans, E. siegelli, E. pampeanum, and E. uncinatum (new naming Epichloe=Neotyphodium) for use controlling intestinal parasites in livestock.

18. The loline alkaloid for use according to claim 1 in which the concentration of loline alkaloid is from 250-25000 μg/g.

19. A grass species infected with an endophyte strain according, to claim 17, wherein the pass species is selected from the group comprising: Festulolium sp, Lolium perenne, Festuca pratense (syn. Lolium pratense (Buds.) Darbysh., and Schedonorus pratensis (Huds.) P.Beauv.)), Festuca arundinacea (syn., Lolium arundinaceum Derbyshire; Schedonorus phoenix (Scop.) Holub), Lolium multiflorum Lam., Lolium temulentum, Festuca arundinacea, Adenocarpus, Convolulacea, Bromus auleticus and hybrids and/or combinations between these species, for use in the treatment of livestock for internal parasitic infestation.

20. Cloned plants, root, tissue and seeds of endophyte containing plants as claimed in claim 17, for use in the treatment of livestock for internal parasitic infestation.
